(19) United States
(12) Patent Application Publication
(10) Pub. No.: US 2026/0223855 A1
(43) Pub. Date: Aug. 6, 2026

(54) DISINFECTANT SOLUTION AND ARTICLE COATED WITH DISINFECTANT SOLUTION

(71) Applicants: Renden TASHIRO, Gifu (JP); Shogo KAWAI, Aichi (JP); Tracy M HEIMS, Seattle, WA (US)

(72) Inventors: Renden TASHIRO, Gifu (JP); Shogo KAWAI, Aichi (JP); Tracy M HEIMS, Seattle, WA (US)

(21) Appl. No.: 18/754,104

(22) PCT Filed: Jun. 26, 2020
(86) PCT No.: PCT/US2020/039717

§ 371 Date: Jun. 25, 2024

(51) Int. Cl. A01N 59/00; A01N 25/02; A01P 1/00; A01P 17/00

(52) U.S. Cl. CPC A01N 59/00; A01N 25/02; A01P 1/00; A01P 17/00

(57) ABSTRACT

To provide a disinfectant solution suitable for various objects to be coated that are normally seen in daily life rather than in a special environment. The disinfectant solution of the present invention includes stabilized chlorine dioxide having an immediate effect on pathogens, a repellent substance or an antibacterial substance each having a slow-acting effect on pathogens, and a surfactant that allows the stabilized chlorine dioxide to blend with the repellent substance or the antibacterial substance.

### TECHNICAL FIELD

The present invention relates to a disinfectant solution and an article coated with the disinfectant solution, and more particularly to a disinfectant solution for a door knob, a handle, an operation button for home electric appliances, a bank ATM, other electronic devices with keypads, a touch panel display such as a ticket vending machine, and the like, and an article coated with the disinfectant solution.

### BACKGROUND ART

Patent Literature 1 discloses a liquid composition having antibacterial properties, which does not easily discolor or lose antibacterial properties even when exposed to UV or chloride ions. The liquid composition is a liquid composition having antibacterial properties which is formed by mixing at least a solvent, one or more compounds selected from the group consisting of organic acid silver salt and inorganic acid silver salt, each of which does not have an amine structure in its molecule and is soluble in the solvent, a cationic polymer being one or more compounds selected from the group consisting of a polycation, a basic peptide, and a salt thereof, each of which has an amine structure in its molecule and is soluble in the solvent, whereby a silver complex formed by using the cationic polymer as a silver ion ligand is soluble in the solvent.

### CITATION LIST

### Patent Literature

Patent Literature 1: JP 2020-007277 A

### SUMMARY OF INVENTION

### Technical Problem

However, as for the use of the liquid composition disclosed in Patent Literature 1, the main focus is on the fact that even if an object is exposed to UV or chloride ions, discoloration occurs, or antibacterial properties are impaired. Unlike that, repellent performance and antibacterial performance are less focused.

In particular, in a case where pathogens have come into contact with a certain object to be coated, some objects to be coated are required to reduce the number of pathogens in a short period of time, and some objects to be coated are required to reduce the number of pathogens for a long period of time. In this way, the specs demanded for the disinfectant solution vary.

Therefore, one of the main objectives of the present invention is to provide a disinfectant solution suitable for various objects to be coated that are normally seen in daily life rather than in a special environment.

### Solution to Problem

In order to solve the above objective, the disinfectant solution of the present invention includes:
stabilized chlorine dioxide having an immediate effect on pathogens;
a repellent substance or an antibacterial substance each having a slow-acting effect on pathogens; and
a surfactant that allows the stabilized chlorine dioxide to blend with the repellent substance or the antibacterial substance.

The stabilized chlorine dioxide can be chlorous acid or a compound thereof, or perchloric acid or a compound thereof. Specifically, as the stabilized chlorine dioxide, for example, some of chlorous acid, sodium chlorite, magnesium chlorite, potassium chlorite, calcium chlorite, ammonium chlorite, barium chlorite, barium subchloride, perchloric acid, sodium perchlorate, magnesium perchlorate, potassium perchlorate, calcium perchlorate, ammonium perchlorate, and barium perchlorate can be used.

A concentration of the stabilized chlorine dioxide can be, for example, in a range of 50 ppm to 500 ppm, and a concentration of the repellent substance or a concentration of the antibacterial substance can be in a range of 10 ppm to 500 ppm.

The repellent substance can be at least one or more of 2-mercaptopyridine N-oxide sodium salt (CAS RN=3811-73-2), N,N-diethyl-3-methylbenzamide (CAS RN 134-62-3), and 2-(2-hydroxyethyl)-1-piperidinecarboxylic acid, 1-methylpropyl ester (CAS RN 119515-38-7).

Further, the antibacterial substance can be at least one or more of polyhexamethylene bigranide (CAS RN=3228-58-0), N-acetyl-N-butyl-β-alanine ethyl ester (CAS RN=52304-36-6), 2-(methoxycarbonylamino)-1H-benzimidazole (CAS RN=10605-21-7), tetradecyl[3-(trimethoxysilyl)propyl]dimethylaminium chloride (CAS RN=41591-87-1), N-(trichloromethylthio)-4-cyclohexene-1,2-dicarboxylimide (CAS RN 133-06-2), N-(2,3-dichloro-4-hydroxyphenyl)-1-methylcyclohexanecarboxamide (CAS RN 126833-17-8), and 2-(4-thiazolyl)-benzimidazole (CAS RN 148-519-8).

The article of the present invention is coated with the disinfectant solution. The article referred herein is an object to be coated with the disinfectant solution, and the object is usually seen in daily life, such as door knobs, various buttons, electric appliances, and kitchen appliances, parts that people touch when opening and closing window glass, or parts that people touch when opening and closing a car door.

### DESCRIPTION OF EMBODIMENTS

Hereinafter, embodiments of the present invention will be described. The term “antibacterial” in this specification includes antifungal and antivirus. The disinfectant solution of this embodiment has an antibacterial effect.

### Composition of Disinfectant Solution

The disinfectant solution of this embodiment contains, as described below, stabilized chlorine dioxide, a repellent substance and/or an antibacterial substance, a surfactant, and an additive.

Stabilized chlorine dioxide has an immediate effect on pathogens such as bacteria, molds, and viruses, and is most contained in the water constituting the disinfectant solution. The stabilized chlorine dioxide can be chlorous acid or a compound thereof, or perchloric acid or a compound thereof. The immediate effect as used herein means that an antibacterial activity against the contacted pathogens is recognized within a few tens of seconds.

Specific examples of stabilized chlorine dioxide that can be used for the disinfectant solution of this embodiment include chlorous acid, sodium chlorite, magnesium chlorite, potassium chlorite, calcium chlorite, ammonium chlorite, barium chlorite, barium subchloride, perchloric acid, sodium perchlorate, magnesium perchlorate, potassium perchlorate, calcium perchlorate, ammonium perchlorate, and barium perchlorate. The concentration of stabilized chlorine dioxide may be in a range of 50 ppm to 500 ppm.

Here, an advantage of producing a disinfectant solution using stabilized chlorine dioxide is that, like a disinfectant solution prepared by using sodium hypochlorite, a disinfectant action is obtained, but dioxide is not generated.

That is, when any of the above disinfectant solutions comes into contact with pathogens, a disinfectant action of destroying cell membranes of the pathogens is given by an oxidation reaction. In the case of the disinfectant solution prepared by using sodium hypochlorite, at this time, dioxide is generated due to separation between oxygen and salt. However, in the case of the disinfectant solution prepared by using stabilized chlorine dioxide, although it is a chlorine-based, the separation between oxygen and salt does not occur and dioxide is not generated. This is advantageous.

The repellent substance has an antibacterial effect not only on the surface of the object to be coated, but also on the periphery of the object to be coated even when the object to be coated is a cloth such that the disinfectant solution penetrates. When such an antibacterial substance is used, the antibacterial effect of the repellent substance can be obtained even if the surface of the object to be coated is touched by human hands.

Specific examples of the repellent substances include 2-mercaptopyridine N-oxide sodium salt (CAS RN=3811-73-2), N, N-diethyl-3-methylbenzamide (CAS RN 134-62-3) , and 2-(2-hydroxyethyl)-1-piperidinecarboxylic acid, 1-methylpropyl ester (CAS RN 119515-38-7). These repellent substances may be used singly, or as a mixture of a plurality of kinds thereof.

The antibacterial substance has an antibacterial effect on a surface of an object to be coated. When the antibacterial substance is used, as long as it is located on the surface of the object to be coated, an antibacterial effect can be obtained even if the surface is touched by human hands.

Specific examples of the antibacterial substance that can be used for the disinfectant solution of this embodiment include antibacterial substances containing some of antibacterial components such as polyhexamethylene bigranide (CAS RN=3228-58-0), N-acetyl-N-butyl-β-alanine ethyl ester (CAS RN=52304-36-6), 2-(methoxycarbonylamino)-1H-benzimidazole (CAS RN=10605-21-7), tetradecyl[3-(trimethoxysilyl)propyl]dimethylaminium chloride (CAS RN=41591-87-1), N-(trichloromethylthio)-4-cyclohexene-1,2-dicarboxylimide (CAS RN 133-06-2), N-(2,3-dichloro-4-hydroxyphenyl)-1-methylcyclohexanecarboxamide (CAS RN 126833-17-8), and 2-(4-thiazolyl)-benzimidazole (CAS RN 148-519-8), however these vary depending on the object to be coated.

In the simplest way, the repellent substances and the antibacterial substances may be grouped in accordance with all the above-described exemplifications or the same attributes, and any of the substances belonging to the groups may be mixed in the same amount. Then, there is an advantage that antibiotics against various bacteria can be obtained by the action produced by each antibacterial component. The concentration of the repellent substance or the concentration of the antibacterial substance may be in a range of 10 ppm to 10,000 ppm. Unlike the stabilized chlorine dioxide, the repellent substance or the antibacterial substance can have an antibacterial activity against pathogens, which is a slow-acting effect thereon. The slow-acting effect as used herein means that the antibacterial activity against the contacted pathogens is recognized after a few minutes to several tens of minutes.

The surfactant is a substance that allows the stabilized chlorine dioxide to blend with the repellent substance or the antibacterial substance. Even if a solvent, the repellent substance, and the antibacterial substance have different masses and properties, a desired mixed state of the solvent, the repellent substance, and the antibacterial substance can be maintained until the disinfectant solution is dried. As the surfactant, a cationic surfactant, a non-ionic surfactant or the like can be used. The surfactant may be appropriately selected within the range of 1 wt % to 10 wt % with respect to the whole disinfectant solution.

An antioxidant or the like that can provide stability during storage of the disinfectant solution can be used as the additive.

### Method of Producing Disinfectant Solution

First, about 900 mL of distilled water is added to a stirring container. Then, as stabilized chlorine dioxide, for example, chlorous acid is added in an amount of about 10 ppm to 500 ppm, and further the repellent substance and/or the antibacterial substance as described above are added in a total amount of about 10 ppm to 10,000 ppm. While keeping the stirring container at room temperature, the mixture is stirred for about 30 seconds to 60 seconds using an ultrasonic disperser or the like, thereby producing a mixed solution 1.

Subsequently, to the mixed solution 1, cation as a surfactant is added in an amount of about 5 wt % of the finished product of the disinfectant solution. While keeping the inside of the stirring container at room temperature, the resultant mixture is stirred, for example, for about 30 seconds to 60 seconds using an ultrasonic disperser or the like, and thus about 1 L of the disinfectant solution of this embodiment is completed.

Note that it is not essential to perform the appropriate stirring process at room temperature; that means that it is not necessary to perform the cooling or heating process intentionally, and the stirring process under these environments is not excluded.

Articles that are normally seen in daily life such as door knobs, various buttons, electric appliances, and kitchen appliances can be used as the object to be coated with the disinfectant solution of this embodiment.

As described above, the disinfectant solution described in the embodiments is not limited to the scope disclosed in this specification, and includes equivalents according to the spirit of the claims.

1. A disinfectant solution comprising:
stabilized chlorine dioxide having an immediate effect on pathogens;
a repellent substance or an antibacterial substance each having a slow-acting effect on pathogens; and
a surfactant that allows the stabilized chlorine dioxide to blend with the repellent substance or the antibacterial substance.

2. The disinfectant solution according to claim 1, wherein
the stabilized chlorine dioxide is chlorous acid or a compound thereof, or perchloric acid or a compound thereof.

3. The disinfectant solution according to claim 1, wherein
the stabilized chlorine dioxide is some of
chlorous acid, sodium chlorite, magnesium chlorite, potassium chlorite, calcium chlorite, ammonium chlorite, barium chlorite, barium subchloride,
perchloric acid, sodium perchlorate, magnesium perchlorate, potassium perchlorate, calcium perchlorate, ammonium perchlorate, and barium perchlorate.

4. A disinfectant solution, wherein a concentration of the stabilized chlorine dioxide is in a range of 50 ppm to 500 ppm, and a concentration of the repellent substance or a concentration of the antibacterial substance is in a range of 10 ppm to 500 ppm.

5. The disinfectant solution according to claim 1, wherein the repellent substance is at least one or more of 2-mercaptopyridine N-oxide sodium salt (CAS RN=3811-73-2), N,N-diethyl-3-methylbenzamide (CAS RN 134-62-3), and 2-(2-hydroxyethyl)-1-piperidinecarboxylic acid, 1-methylpropyl ester (CAS RN

6. The disinfectant solution according to claim 1, wherein the antibacterial substance is at least one or more of polyhexamethylene bigranide (CAS RN=3228-58-0), N-acetyl-N-butyl-β-alanine ethyl ester (CAS RN=52304-36-6), 2-(methoxycarbonylamino)-1H-benzimidazole (CAS RN=10605-21-7), tetradecyl[3-(trimethoxysilyl)propyl]dimethylaminium chloride (CAS RN=41591-87-1), N-(trichloromethylthio)-4-cyclohexene-1,2-dicarboxylimide (CAS RN 133-06-2), N-(2,3-dichloro-4-hydroxyphenyl)-1-methylcyclohexanecarboxamide (CAS RN 126833-17-8), and 2-(4-thiazolyl)-benzimidazole (CAS RN 148-519-8).

7. An article coated with the disinfectant solution according to claim 1.
